(19) United States
(12) Patent Application Publication
(10) Pub. No.: US 2006/0165766 A1
(43) Pub. Date: Jul. 27, 2006

(54) Method for preparing liposome formulations with a predefined release profile

(76) Inventors: Yechezkel Barenholz, Jerusalem (IL); Veronica Wasserman, Thornhill (CA)

Correspondence Address: NATH & ASSOCIATES, 112 South West Street, Alexandria, VA, 22314, US

(21) Appl. No.: 10/488,468

(22) PCT Filed: Sep. 9, 2002
(86) PCT No.: PCT/IL02/00745

§ 371 Date: Jan. 7, 2005

Related U.S. Application Data
(60) Provisional application No. 60/317,187, filed on Sep. 6, 2001.

(51) Int. Cl. A61K 9/127

(52) U.S. Cl. 424/450

(57) ABSTRACT

The present invention provides a novel tool for designing a release profile of an active agent from a liposome in to which it is loaded. According to the invention, a method is provided for preparing a liposomal formulation for delivery of an active agent to a target, the release of said active agent from the liposome being designed to have a release profile in which the release is sustained for a time period to achieve an optimal effect of the active agent at said target, the method comprising preparing a liposomal formulation, wherein the liposome is loaded with said active agent and with a selected counter ion, the counter ion and the active agent interacting together to aggregate and/or to form a precipitate within the liposome, the counter ion being selected such that the release of the active agent from the liposome has said release profile.

### FIELD OF THE INVENTION

The present invention relates in general to liposome formulations in which the active agent encapsulated by the liposome is released therefrom according to a predefined release profile.

### LIST OF PRIOR ART

1. U.S. Pat. No. 5,316,771;
2. U.S. Pat. No. 5,192,549;
3. U.S. Pat. No. 5,939,096;
4. U.S. Pat. No. 6,162,462;
5. Barenholz Y, In: Medical Applications of Liposomes (Lasic, D. D. and Papahadjopoulos, D., eds.), Elsevier Science, Amsterdam, pp. 541-565 (1998).
6. Barenholz Y, Curr. Opin. Colloid Interface Sci. 6:66-77 (2001).
7. PCT publication No. WO 00/66126

### BACKGROUND OF THE INVENTION

Liposomes were first described nearly 40 years ago and have been useful models for studying the physical chemistry of lipid bilayers and the biology of the cell membrane. It was also realized that liposomes might be used as vehicles for the delivery of drugs and other active agents as well as in the field of gene transfer.

Liposome technology faces two main challenges. The first challenge is to achieve a high level of loading of an active agent in the liposome and to make that loading stable during handling and storage. The second is to be able to fit the release rate of the loaded/associated active agent to specific aims of the liposome formulation.

Loading of an agent into liposomes has proven to be a measure of their utility. A poor liposome loading, leads to low drug/lipid ratio, and therefore the use of liposomes as a vehicle becomes inefficient at the target site. In addition, with poor loading, there is a great loss of the active agent which makes liposome-based drugs uneconomical.

So far, several methods have been developed for loading agents into liposomes. The simplest method of agent loading includes a passive entrapment of water soluble agents during a dry lipid film by hydration of the lipid components. The loading efficiency of this method is generally low as it depends on the entrapping volume of the liposome, on the concentration of the drug and its solubility in the hydration medium as well as on the amount of lipids used to prepare them.

Improved passive entrapment of an agent into liposome has been achieved by using a dehydration-rehydration method according to which preformed liposomes are added to an aqueous solution containing the agent, followed by dehydration of the mixture, by lyophilization, evaporation or by freeze-thaw processing method. This is explained by increase in trapped aqueous volume and equilibrating of the solute throughout all the intraliposomal aqueous phase.

Loading of an agent into liposomes may also be achieved by the use of high lipid concentration or by the use of a specific combination of lipid components.

A method of encapsulating hydrophilic agents involves reverse evaporation from an organic solvent. According to this approach, a mixture of a hydrophilic agent and vesicle-forming lipids are emulsified in a water-in-oil emulsion, followed by solvent removal to form an unstable lipid-monolayer gel. When the gel is agitated, typically in the presence of added aqueous phase, the gel collapses to form oligolamellar liposomes with high efficiency of encapsulation of the agent. However, the limitation of agent solubility and trapped volume still applies.

In the case of ionizable hydrophilic or amphipathic agents loading can be achieved by forming a transmembrane pH gradient. Typically, the agent contains an ionizable amine group, and is loaded by adding it to a suspension of liposomes prepared to have an inside/outside pH gradient. For example, when using an ammonium gradient, ammonium within the liposomes are in equilibrium with ammonia, which is freely permeable through the liposome membrane, and protons, which therefore accumulate, as ammonia is lost from the liposomes, leading to the lower inside/outside pH gradient. After establishing the gradient, excess ammonium ions within the liposomes provide a reservoir of protons, to maintain the liposome pH gradient over time.

The release rate of the loaded molecule from liposomes was shown to be dependent on: temperature, medium-related properties (medium composition, ionic strength, pH), liposome-related properties (membrane lipid composition, liposome type, number of lamellae, liposome size, physical state of phospholipid membrane i.e., liquid-disordered (LD), liquid-ordered (LO), solid-ordered (SO)), and loaded-molecule-related properties (lipophilicity, hydrophilicity, size) [Haran G., et al., Biochim Biophys. Acta 1151:201-215, (1993)].

The present invention aims for providing a tool for designing a release profile of an active agent, e.g. a drug, such that the agent is released from liposomes in which it is encapsulated according to predetermined release rates.

### SUMMARY OF THE INVENTION

Thus, according to the present invention there is provided a method for preparing a liposomal formulation for delivery of an active agent to a target, the release of said active agent from the liposome into which it is loaded being designed to have a release profile such that the release is sustained for a time period to achieve a desired optimal effect of the active agent at said target, the method comprising:

preparing a liposomal formulation, wherein the liposome is loaded with said active agent, and with a selected counter ion, said counter ion and said active agent interacting together, to form an aggregate and/or to form a precipitate within the liposome, the counter ion being selected such that the release of the active agent from the liposome has said release profile.

The formulation according to the invention may have various applications, including therapeutical, nutritional, or environmental applications as well as others. Evidently, this will depend, inter alia, on the active agent, the type and concentration of the ingredients forming the liposomal formulation and the specific release profile designed, as well as on other factors known to those versed in the art.

The term “release profile” as used herein refers to the characteristics of the release of the active agent from the liposome onto which it is loaded and will be designed according to the specific application of the formulation obtained. The term release profile encompass any type of controlled release profile, including: delayed, sustained or prolonged release, gradual release, timed release, pH dependent release etc. The selection of the desired release profile will depend on considerations known to the artisan, such as the condition and location of the target to be treated, the purpose of application of the formulation (therapeutic etc.), the treatment regime, etc.

The term “ache agent” as used herein refers to a molecule which biologically or chemically acts on the selected target. According to one embodiment, the active agent is a drug acting on a desired target cell or tissue. According to another embodiment, the active agent is a molecule (e.g. low molecular weight compound) which chemically reacts at its target to result in a chemical effect.

The term “target” used herein refers to any target on which an active agent is designed to act. The target is preferably a localized site such as a specific target cell or tissue within a living body. However, at times, the formulation of the invention may be designed for environmental purposes, such as for treating contaminated water, for treating aquariums, etc. For example, the active agent may be an anti-chlorine agent to remove from the aqueous medium chlorine.

As used herein the term “liposome” is intended to include all spheres or vesicles comprised of liposome-forming substances. These are such that spontaneously or non-spontaneously vesiculate, and include particularly amphipathic substances; such as phospholipids, which are glycerides in which at least one hydrocarbon chain (an acyl or alkyl) is are replaced by a complex phosphoric acid ester.

As used herein, the term “loading” is intended to include any kind of interaction between the active agent and the liposome, for example, an interaction such as encapsulation, adhesion, adsorption, entrapment, (to the inner or outer wall of the vesicle or in the intraliposomal aqueous phase) or embedment of the active agent in the liposome's membrane, with or without extrusion of the liposome containing the active agent. Preferably, according to the present invention, loading refers to intraliposomal encapsulation.

Finally, the terms “aggregate” or “precipitate” concern any type of chemical or physical association between the active agent and the counter ion, both loaded into the liposome, to form a salt. The formation of the salt leads to the formation of an insoluble product (which may result in the formation of a precipitate), or to the formation of an aggregate product. In any case, the counter ion may be in a free form or covalently attached to a water-soluble polymer such as dextrone, arabino galactan and others.

According to the present invention the level of interaction (chemical association) between the active agent and the counter ion may be controlled by the selection of the counter ion, such that for different release profiles, different counter ions are selected thereby providing different levels of interactions, each of which correlate with a different, predefined, release profile. It may be the case that no interaction/precipitation occurs between the active agent and the counter ion, in which case, no or substantially no aggregates are formed and the resulting release profile obtained, will define a substantially fast release rate of the agent from the liposome.

### BRIEF DESCRIPTION OF THE FIGURES

In order to understand the invention and to see how it may be carried out in practice, some embodiments will now be described, by way of non-limiting examples only, with reference to the accompanying figures, in which:

FIG. 1A-1C represent the osmolality calibration curves of Tempamine (TMN) (FIG. 1A), Bupivacaine (BUP) (FIG. 1B), and Acridine Orange (AO) (FIG. 1C) in DDW (10-40 mM).

FIG. 2A-2F represent the effect of TMN (25 mM) on osmolality of different salts (∘), including ammonium sulfate (FIG. 2A), ammonium citrate (FIG. 2B), ammonium phosphate (FIG. 2C), ammonium chloride (FIG. 2D), ammonium glucuronate (FIG. 2E) and —NaCl FIG. 2F) as compared to the osmolality of the salts alone (●).

FIGS. 3A-3F represent the contribution of 25 mM acridine orange (●) and 25 mM bupivocaine (∘) to osmolality of different ammonium salts: ammonium sulfate (FIG. 3A), ammonium citrate (FIG. 3B), ammonium phosphate (FIG. 3C), ammonium chloride (FIG. 3D), ammonium glucuronate (FIG. 3E) or NaCl (FIG. 3F). Bold lines represent the osmotic pressure of the specific ammonium salt or NaCl alone (−).

FIGS. 4A-4B represent the stability of TMN remote loading into egg PC MLV (1200±200 nm) at 25° C. (FIG. 4A) and 37° C. (FIG. 4B), with intraliposomal salt concentration of 200 mM.

### DETAILED DESCRIPTION OF THE INVENTION

The present invention is based on the finding that there is a correlation between the level of aggregation/precipitation of an agent, such as amphipathic weak bases or amphipathic weak acids encapsulated in liposome with a counter ion, and the agent's release profile from the liposome.

In particular, it has now been found and is first disclosed herein that by specific selection of a counter ion of a weak base or of a weak acid, the latter being the active agent, and both are encapsulated in a liposome, the release profile of the active agent from the liposome may be controlled. It has now been established that the major parameter dictating the release profile of the active agent from the liposome depends on the extent of chemical association between the agent and the counter ion, i.e. by controlling the ratio between precipitated and unprecipitated active agent (by the use of a selected counter ion) it is possible to dictate the release rate of the agent from the liposome, thus, obtaining a predefined, specifically desired, release profile.

The effect of aggregation on stability of liposomes was previously described [Barenholz, 1998, ibid.; Barenholz, 2001 ibid.], according to which there is a need for extensive aggregation of a drug in order to obtain a stable liposomal formulation with respect to drug leakage. Now, by the present invention, a method for designing a formulation with a predefined release profile is disclosed. In particular, the present invention discloses that specific selection of the counter ion associating with the agent in the liposome is a tool for the design of controlled delivery systems.

In the present invention, a preferred method of preparing the liposomes is the remote loading method. According to this method, the active agent, (in the following specific examples, TMN, AO or BUP) is loaded into liposomes by remote loading against an ion concentration gradient. This method has been previously described, inter alia, in U.S. Pat. No. 5,192,549.

A typical procedure for forming the liposomes involves dissolving a mixture of liposome-forming lipids in a suitable organic solvent and evaporating the organic solvent in a vessel to form a thin film. The film is then covered with an aqueous medium containing the solute species that will form the aqueous phase in the liposome interior spaces. After liposome formation, the vesicles are sized according to known methods (e.g. as sonication) to achieve a size distribution of liposomes with a selected range (preferably uniformly sized).

The liposomes encapsulating the active agent may be prepared as multilamellar vesicles (MLV), by solvent injection, lipid hydration, reverse evaporation, freeze drying or by repeated freezing and thawing. Yet, small (<100 nm) or large (>100 nm) unilamellar vesicles (SUV or LUV, respectively) may be prepared e.g. by sonication, by extrusion through polycarbonate filters having a defined pore size, by using a French pressure cell, i.e., by passing MLV through small orifice under high pressure, or by solvent injection methods, with solvents is such as ethers or alcohols. Other types of vesicles which may be formed include large unilamellar vesicles (LUV); stable plurilamellar vesicles (SPLV), oligolameliar vesicles (OLV) whether prepared by detergent removal using dialysis, column chromatography, bio-beads SM-2, by reverse phase evaporation (REV); intermediate sized unilamellar vesicles formed by high pressure extrusions [Methods in Biochemical Analysis, 33:337 (1988)] or giant multivesicular vesicles (GMVV, U.S. Pat. No. 6,162,462) liposomes, at least 1 microns in diameter, prepared by vortexing a lipid film with an aqueous solution of a suitable salt (e.g. ammonium sulfate), homogenizing the resulting suspension to form a suspension of small unilamellar vesicles (SUV), and repeatedly freeze-thawing said suspension of SWV in liquid nitrogen followed by water to form the GMVV. All these and other methods of liposome preparation, known in the art, are useful in practicing the present invention. These methods are described in U.S. Pat. Nos. 4,235,871; 4,241,046; 4,529,561; 4,737,323 and 4,752,42, 5,316,771 and 6,162,462, incorporated herein by reference.

After sizing, the external medium of the liposomes is treated to produce an ion or pH gradient across the liposome membrane, which is typically a lower inside/higher outside concentration gradient This may be achieved by a variety of methods including (i) diluting the external medium, (ii) dialysis against the desired final medium, (iii) molecular-sieve chromatography, for example, using Sephadex G-50, against the desired medium, or (iv) high-speed centrifugation and resuspension of the pelleted liposomes in a desired final medium. The external medium which is selected will depend on the mechanism of gradient formation and the external pH desired, as will now be considered.

According to one preferred embodiment, an ion gradient (also referred to as a proton gradient) is produced by creating an ammonium ion gradient across the liposome membrane, as described, for example, in U.S. Pat. Nos. 5,192,549 and 5,316,771, incorporated herein by reference. The liposomes are prepared in an aqueous buffer containing an ammonium salt, such as those employed herein is (ammonium sulfate, ammonium phosphate, ammonium citrate, etc.), or by the use of sulfated polymers such as dextran ammonium sulfate or heparin sulfate, the buffer adjusted to a suitable pH. After liposome formation and sizing, the external medium is replaced with a medium lacking ammonium ions, for example, with NaCl or a sugar at a concentration that gives a similar osmolality inside and outside of the liposomes (although, at times, a greater outer osmolarity may be employed), and the ammonium ions inside the liposomes are in equilibrium with the ammonia and protons. The up-protonated ammonia is able to penetrate the liposome bilayer and escape from the liposome interior which continuously shifts the equilibrium, within the liposome.

Alternatively, the aqueous hydration medium may contain a polymer to which the counter ion is covalently attached. Such charged polymers are used as macro counter ions that improve the control of release rate of drug from liposomes. To this end, anionic polymers may be used to interact with amphipathic weak bases, and cationic polymers, to interact with amphipathic weak acids. In case of polymeric counter ions, an agent is loaded into a liposome by a gradient such as pH gradient, ammonium gradient or acetate gradient to fit loading of amphipathic weak bases or acids [Barenholz Y (2001); Haran G., et al., (1993) ibid.; Clerc S. and Barenholz Y, Biochim Biophys Acta. 1240(2):2576 (1995)]. Inside the liposome the agent is ionized due to the pH and/or ion gradient and forms a salt with the low molecular weight counter ion (e.g. PO₄⁻³, SO₄⁻² and the like for weak bases, and Na⁺, Ca⁺², Mge, Ba⁺², Al⁺³ and the like for weak acids). After permeation into the intraliposomal aqueous phase and ionization, the agent interacts with the counter ion of the polymer (e.g., for bupivacaine as the agent, it may interact with the sulfate moiety of the polymer dextran sulfate). The salt thus formed between the two constituents induces aggregation of a polymer-agent salt inside the liposome aqueous phase. This aggregation, although reversible, acts as depot for the agent. The factors that determine rate of agent release include: the balance between the level of polymer charged groups (charges/mg polymer), the dissociation constant of the charged group, the association constants between the agent and the polymer, the type and the concentration of the low molecular weight counter ion, and the concentration of the species responsible for the gradient leading to agent loading, together with the permeability coefficient of the agent. For example dextran sulfate of 10,000 D may bind up to 50 nmoles of an amphipathic weak base/molecules (such as acridine orange and bupivacaine) per one molecule of the polymer.

The active agent is loaded into the liposomes by its addition to a suspension of the ion gradient liposomes under conditions effective to allow passage of the active agent from the external medium into the liposomes. Effective passage is such which allows diffusion of an uncharged form of the active agent into the liposomes, which leads to high concentration of the agent loaded within the liposome.

Liposomes are formed from amphipathic compounds, which may spontaneously or non-spontaneously vesiculate. Such amphipathic compounds typically include triacylglycerols or trialkylglycerols where at least one acyl or one alkyl group is replaced by a polar and/or changed moiety, e.g. phospholipids formed by a complex phosphoric acid esters. Any commonly known liposome-forming lipids are suitable for use by the method of the present invention. The source of the lipid or its method of synthesis is not critical: any naturally occurring lipid, with and without modification, or a synthetic phosphatide can be used.

The lipidic substance may be any substance that forms liposomes upon dispersion thereof in an aqueous medium. Preferred liposome-forming amphipathic substances are natural, semi-synthetic or fully synthetic, molecules; negatively or positively charged lipids, phospholipids or sphingolipids, optionally combined with a sterol, such as cholesterol; and/or with lipopolymers, such as PEGylated lipids.

In particular, the of vesicle-forming lipids may include dialiphatic chain lipids, i.e. phospholipids as indicated above, diglycerides, dialiphatic glycolipids, lipids such as sphingomyelin and glycosphingolipid, cholesterol derivatives, alone or in combinations and/or with or without liposome membrane rigidifying agents.

“Phlospholipis” are triacyl, trialkyl (or their combination) lipids in which at is least one acyl or alkyl group is replaced by a complex phosphoric acid ester and include, inter alia, phosphatidic acid (PA) and phosphatidylglycerol (PG), phosphatidylcholine (PC), phosphatidylethanolamine (PE), phosphatidylinositol (PI), phosphatidylserine (PS), plasmalogens, phosphatidic acid, sphingomyelin soybean derived phospholipids, egg yolk phospholipids and derivatives such as dipalmitoylphosphatidylcholine (DPPC), dimyristoyl phosphatidylcholines (DMPC), dimyristoyl phosphatidyl-choline (DMPG), egg phosphatidylcholine (EPC), partially hydrogenated egg phosphatidylcholine (PHEPC), distearylphosphatidylcholine (DSPC), hydrogenated soy PC(HSPC) splingomyelin and others. These phospholipids have varying degrees of saturation and may be fully saturated or partially hydrogenated. The source of the phospholipid or its method of synthesis are not critical, any natually occurring, semisynthetic or synthetic phosphatide can be either obtained commercially or prepared according to published methods.

In the dialiphatic chain lipids, which preferably make up the bulk of the vesicle-forming lipids, the aliphatic chains are preferably at least about 12 carbon atoms in length, and optimally are between about 14 and 24 carbon atoms long. The chains are also partially or substantially saturated, by which is meant that each chain may contain one unsaturated (double) bond. As will be appreciated by those versed in the art, the saturated aliphatic chains produce better lipid packing in the liposomes and substantially extend the stability of the liposome formulations by eliminating lipid oxidativeiperoxidative lipid damage. This lack of oxidative damage is observed even in the absence of lipophilic free-radical quenchers, such as α-tocopherol (vitamin E) or butylated hydroxytoluene (BHT), which, and any other lipid protective agents, may be optionally added in effective amounts to the formulation.

The liposome may further include other suitable lipids, such as glycolipids or sterols, such as cholesterol, cholesteryl hemisuccinate, cholesteryl sulfate, other derivatives of cholesterol, lipoproteins (e.g. pegylated lipids), glycosphingolipids (e.g. gangliosides). The liposome may be further formulated to include minor amounts of fatty alcohols, fatty acids, and/or cholesterol esters or any other pharmaceutically acceptable excipients which affect the surface charge, the membrane fluidity and increase the incorporation of the active ingredient in the liposomes.

The term “glycolipid” as used herein is intended to encompass in case of sphingoglycolipids, lipids having two hdyrocarbon chains one of which is the hydrocarbon chain of sphingosine, the other is an acyl chain, and one or more sugar residues attached to the sphingosine. Examples of sphingoglycolipids suitable for practice of the present invention include cerebrosides, galactocerebrosides, glucocerebrosides, GM₁, sulfatides and sphingolipids with di- and tri-saccharides as their polar head groups, i.e. di- and tri-hexosides.

Other glycolipids are the glyceroglycolipids which resemble phospholipids however, their head-group (which may or may not contain a phosphate group) always contain carbohydrate moieties.

Cationic lipids (mono or polycationic) are also suitable as liposome-forming substances. Cationic lipids typically consist of a lipophilic moiety, such as a sterol or the same glycerol backbone to which two acyl or two alkyl, or one acyl and one alkyl chains contributed the hydrophobic region of the amphipathic molecule, to form a lipid having an overall net positive charge. Preferably, the head group of the lipid carries the positive charge. For example, mono cationic lipids include 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N-(N′,N′-dimethylaminoethane) carbamoly] cholesterol (DC-Chol); and dimethyl-dioctadecylammonium (DDAB). Examples of polycationic lipids include a similar lipopholic part as with the mono cationic lipids, to which spermine or spermidine are attached such as N-[2-[[2,5-bisβ-aminopropyl)amino]-1-oxopentyl]amino]ethyl]-N,N-dimethyl-2,3-bis[(1-oxo-9-octadecenyl)oxy]-1-propanaminium (DOSPA), which may be used either alone or in combination with cholesterol or with neutral phospholipids. The cationic lipids may form part of a derivatized phospholipids such as the neutral lipid dioleoylphosphatidyl ethanolamine (DOPE) derivatized with polylysine to form a cationic lipopolymer.

The liposomes may also include a lipopolymer, which is diacly, dialkyl or acylalkyl glycerol groups (or ceramide) derivatized with a hydrophilic polymer. As has been described, for example in U.S. Pat. Nos. 5,013,556, 5,817,856 and 6,165,201 and in WO 98/07409, such a hydrophilic polymer provides a surface coating of hydrophilic polymer chains on both the inner and outer surfaces of the liposome lipid bilayer membranes. The outermost surface coating of hydrophilic polymer chains is effective to provide a liposome with a long blood circulation lifetime in vivo. The inner coating of hydrophilic polymer chains extends into the aqueous compartments in the liposomes, between the lipid bilayers and into the central core compartment, and is in contact with any entrapped agents.

Hydrophilic polymers suitable for derivatization with a vesicle-forming diacyl glycerol or ceramide lipid include polyvinylpyrrolidone, polyvinylmethylether, polymethyloxazoline, polyethyloxazoline, polyhydroxypropyloxazoline, polyhydroxypropylmethacryl-amide, polymethacrylamide, polydimethylacrylamide, polyhydroxypropyl-methacrylate, polyhydroxyethylacrylate, hydroxymethylceliulose, hydroxy-ethylcellulose, polyethyleneglycol, and polyaspartamide. The polymers may be employed as homopolymers or as block or random copolymers. A preferred hydrophilic polymer chain is polyethyleneglycol (PEG), which when combined with a lipid forms what is referred to herein as a PEGylated lipids.

“PEGylated lipids” as used herein refer to combination products of polyethylene oxides lipids, to form lipopolymers. The polyethylene oxides, are preferably polyethers of molecular weight between 500 and 20,000 Daltons more preferably between about 500 and about 5,000 Daltons, most preferably between about 1,000 to about 5,000 Daltons. Membranes of PEG-liposomes typically have different properties from membranes of solely phospholipid liposomes. Methoxy or ethoxy-capped analogues of PEG are also preferred hydrophilic polymers, commercially available in a variety of polymer sizes, for example, with a molecular weight in the range of 120-20,000 g/mol.

Preparation of such vesicle-forming lipids derivatized with hydrophilic polymers has been described, for example in U.S. Pat. Nos. 5,013,556; 5,395,619; 5,817,856; 6,043,094 and 6,165,501, the latter describing lipopolymers containing detachable PEG moieties.

The vesicle-forming lipid can be selected to achieve a specified degree of fluidity or rigidity, to control the stability of the liposome, in the target medium, e.g. in serum and to control the rate of release of the agent entrapped in the liposome. Liposomes having a more rigid lipid bilayer, in the gel (solid ordered) phase or in a liquid crystalline fluid (liquid disordered) bilayer, are achieved by incorporation of a relatively rigid lipid, for example, a lipid having a relatively high phase transition temperature, such as, above room temperature. Rigid, i.e., saturated, lipids having long acyl chains, contribute to greater membrane rigidity in the lipid bilayer. Other lipid components, such as cholesterol, are also known to contribute to membrane rigidity in lipid bilayer structures especially to reduce membrane free volume thereby reducing membrane permeability.

Accordingly, high lipid fluidity is achieved by incorporation of a relatively fluid lipid, typically one having a lipid phase with a relatively low liquid to liquid-crystalline phase transition temperature, for example, at or below room temperature, more preferably, at or below the target body temperature.

The active agent loaded in liposomes using the method of the invention is preferably an amphipathic weak basic/acid substance. Weak basic substances include among others the following active agents: doxorubicin, epirubicin, daunorubicin, carcinomycin, N-acetyladriamycin, rubidazone, 5-imidodaunomycin, N-acetyldaunomycine, all anthracyline drugs, daunoryline, topotecn, irinotecan propranolol, pentamidine, dibucaine, bupivacaine, tetracaine, procaine, chlorpromazine, vinblastine, vincristine, mitomycin C, pilocarpine, physostigmine, neostigmine, chloroquine, amodiaquine, chloroguamide, primaquine, mefloquine, quinine, pridinol, prodipine, benztropinemesylate, trihexyphenidyl hydrochloride, ropranolol, timolol, pindolol, quinacrine, benadryl, promethazine, dopamine, L-OPA serotonin, epinephrine, codeine, meperidine, methadone, morphine, atropine, decyclomine, methixene, propantheline, imipramine, amitriptyline, oxepin, desipramine, quinidine, propranolol, lidocaine, chlorpromazine, romethazine, perphenazine, acridine orange, opiates such as morphine and others.

Weak amphiphatic acids include, without being limited thereto, ibuproten, oluetin, indomethacin, phenylbutazone, mecloferamic acid, piroxicam, citrofloxacin, prostaglandins, fluoresgein, carboxyfluorescein, methyl perdnisolone, and nalidixic acid.

In case of a weak base, the counter ion to be loaded with an active agent into the liposome may be selected from the non-limiting examples including hydroxide, sulfate, phosphate, glucuronate, citrate, carbonate, bicarbonate, nitrate, cyanate, acetate, benzoate, bromide, chloride, and others inorganic or organic anions, or an anionic polymer such as dextrane sulfate, dextrane phosphate, dextane borate, carboxymethyl dextran and the like, while in the case of a weak acid the counter ion may be calcium, magnesium, sodium, ammonium and other inorganic and organic cations, or a cationic polymer such as dextrane spermine, dextrane spermidine, aminoethyl dextran, trimethyl ammonium dextran, diethylaminoethyl dextran, polyethyleneimine dextran and the like. This means that the counter ion may be present in the form of a free small ion or attached to a polymer, or in both forms simultaneously.

The ability of the following three examples of amphipathic weak bases, 2,2,6,6-tetramethylpiperidine-4-amino-1-oxyl (4-amino-Tempo, Tempamine) (T) (an antioxidant), acridine orange (AO) and bupivacaine (BUP, a local anesthetic), to create a complex with different anions of ammonium salts and the level of precipitation/aggregation of the complex was evaluated in the following specific examples.

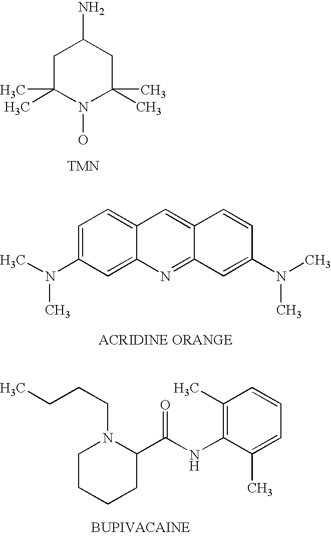

Several publications have already described encapsulation of these amphipathic weak bases in liposomes. For example, BUP may be effectively loaded into liposomes by the formation of ammonium sulfate gradient [Grant et al., Pharm. Res. 18:336-343 (2001); and U.S. Pat. No. 6,162,462). AO is used as a model agent for investigation of loading mechanisms of amphipathic weak bases [Clerc and Barenholz, Anal. Biochem 259:104-111 (1998)] and was shown to aggregate inside liposomes when loaded via an ammonium sulfate gradient. However, as the different resulting precipitates (AO₂SO₄, AONH₄SO₄ or AOHSO₄) were soluble, a high threshold gradient of ammonium sulfate was required in order to achieve the required level of aggregation and therefore, regions in which the drug is only in soluble form could be identified.

Level of aggregation of the exemplified agents was evaluated by measuring osmotic pressure. Osmotic pressure is a colrgative property, depending on the actual concentration (particles per unit volume) of a solution. Thus, in case of aggregate formation in a solution, the actual concentration of the solution will be lower than the theoretical concentration of the solution, which will be evident by a lower than expected osmotic pressure.

In the specific examples remote loading occurs due to pH or ammonium (or ammonim-like, e.g. alkylamine) gradient aggregation due to the high intraliposome concentration of the agent and the formation of agent-sulfate salt. Excess of SO₄⁻² and HSO₄⁻ occurs when the NH₃ is released from the liposomes. Remote loading via an ammonium salt is based on the large difference in permeability of the neutral ammonia gas molecule (1.3×10⁻¹ cm/s) and the charged anion (<10⁻¹⁰ cm/s). Typically, the pH of the intraliposome aqueous phase composed of an ammonium salt solution may be decreased by lowering the external concentration of ammonium and ammonia [Haran, et al., (1993) ibid.]. The decrease of intraliposomal pH results from the release from the liposome of the unprotonated ammonia compound (NH₃) leaving within the liposome protons (H⁺) and sulfate ion (HSO₄⁻, SO₄⁻²) thereby an excess of SO₄⁻² and HSO₄ anions over NH₄⁺ is created within the liposome.

Reduction of the pH inhibits ammonia formation and thereby inhibits its release from the liposome. When adding to the external medium of the liposome an agent, e.g., an amphipathic weak base, it freely crosses the lipid bilayer in its uncharged form and accumulate in its charged (having low permeability) form in the internal aqueous compartment (after being protonated by the free H⁺) [Schuldiner, et al., Eur. J. Bichem 25:64-70 (1972); Nicolas and Deamer, Biochem. Bioplys Acta 455:269-271 (1976)]. Evidently, this accumulation raises the internal pH and thus ammonia is again formed and released from the liposome, resulting in the reduction of internal pH and so forth, until an effective loading of the agent is accomplished.

The equilibrium between charged (protonated) and uncharged agent enables the slow leakage of the uncharged weak base from the liposomes at a rate, which is dependent on the permeability coefficient. Shifting the equilibrium via formation of aggregates (formed between the loaded charged agent and the counter ion within the liposome) further improves the retention of the agent inside the liposome, and as now being disclosed, may function as a tool for controlling the release of the agent from the liposome.

Thus, the present invention enables the design of the release profile of an active agent by controlling the leakage of the un-charged weak base or acid from the liposome. For example, for a release profile to be a slow release profile the counter ion is selected such that its chemical association with said active agent forms within the liposome, a salt with a diminished water solubility (low solubility or substantially insoluble), i.e. a substantially high level of aggregates, and vice versa, for a medium or fast release of the agent, medium or no aggregates are to be formed.

Several ammonium salts were tested in order to evaluate the effect of the counter ion on the release profile of the agent from the liposome, which are: ammonium sulfate, ammonium chloride, ammonium phosphate, ammonium citrate and ammonium glucuronate. These ammonium salts are also those used in order to form the ammonium (pH) gradient in the liposomes. It should be noted that for the formulation of weak amphipathic bases also alkylamines, such as methyl amine, may be employed for forming the required pH gradient (in replace of ammonia). For loading and aggregation of amphipathic weak acids like carboxy fluorescein or methylprednisole, the counter ions: calcium, sodium potassium, barium or aluminium may be used (e.g. derived from calcium formate or calcium acetate).

As will be appreciated, the anions of the above salts have low permeability through the lipid bilayer. For example, the permeability coefficient of Cl⁻ through a phospholipid bilayer is 7.6×10⁻¹¹ cm/s, that of SO₄²⁻ and glucuronate⁻ is <10⁻¹² cm/s, while for dextran sulfate the permeability coefficient is approaching zero.

These salts also differ in the ionic strength of their anion, having the following order: (HSO₄⁻) SO₄⁻², ≈Cl⁻≈HPO₄⁻²(PO₄⁻³)>citrate⁻³>glucuronate⁻, as well as in the charge of the anion.

While the invention will now be described with reference to specific is methods and embodiments, it will be appreciated that various modifications may be made without departing from the invention as defined by the appended claims.

### SPECIFIC EXAMPLES

Materials and Methods

Materials

Ammonium chloride (>99.5% pure), ammonium phosphate, dibasic (>99% pure) and D-glucuronic acid (analytical grade) were obtained from Sigma Chemical Co. (St. Louis, Mo., USA). Ammonium sulfate (99.99% pure) and ammonium citrate, tribasic (99% pure) were supplied by Aldrich Chemical (llwaukee, WI, USA). All salts were prepared in a concentration range of 10-80 mM and at a pH 7.0±0.1 (by titration with ammonium hydroxide or a suitable acid). Ammonium glucuronate was prepared by titration of glucuronic acid solution of a desired concentration with 30% ammonium hydroxide to pH 7±0.1. The free radical, antioxidant, 2,2,6,6-tetramethylpiperidine-4-amino-1-oxyl (4-amino-Tempo, Tempamine (TMN)), 97% pure, was purchased from Aldrich. Acridine orange hemi(zinc chloride) salt (AO, 86% dry dye) (MW=370) was obtained from Sigma Bupivacaine HCl (>99% pure BUP) was obtained from G. J. Grant (NYU School of Medicine, N.Y.).

Methods

Osmolality Measurements

For osmotic pressure measurements, double distilled water (DDW) obtained using Water ProPlus (Labconco, Kansas City, Mo., USA) was used for all solutions. Osmolality was measured using a 5500 Vapor Pressure Osmometer (Wescor Inc., Logan, Utah, USA) or freezing point osmometer μOSMETIE MicroOsmometer of Precision Systems (atich, MA, USA). Calibration curves of TMN, AO, and BUP osmolality in the range of 10-40 mM in pure water were used to describe the relationship between concentration and osmotic pressure of all compounds. Based on these calibration curves a concentration of 25 mM for all agents evaluated was used in the following specific examples, which gave the most reliable measurements.

Conductivity Measurements

Solutions in concentration range of 0.5-50 mM of NaCl and ammonium salts were prepared in DDW and measured in an “Oyster” conductivity meter (KTech).

Liposome Hydration Medium

The different ammonium salts were prepared at 3 different concentrations: 100, 200, and 400 mM and were brought to pH 7.0±0.1 (by titration with ammonium hydroxide or a suitable acid).

Preparation of Liposomes

Multilamellar liposomes (MLV) (1200±200 nm) composed of egg PC were used. To exclude the influence of liposomal membrane on loading stability, liposomes with membranes in a liquid disordered (fluid) state at room temperature were used. Such liposomes are typically leaky, thereby depending the release profile of the active agents properties solely. MLV were formed through one-step hydration in the desired salt medium followed by homogenizing the phospholipid in the hydration medium by a high-shear Polytron homogenizer (Kinematica, Luzern, Switzerland) for several minutes. The distribution of liposome sizes in the preparations was measured by photon correlation spectroscopy using a Coulter (Model N4 SD) sub-micron particle analyzer.

Formation of Gradient

A dialysis procedure developed by Haran et al. [Haran G. et al. (1993) ibid.] was utilized for the formation of the pH gradient. In general, two consecutive as dialysis exchanges against 100 volumes of 0.15 M NaCl (pH=5.2) and a third dialysis exchange against 100 volumes of 0.15 M KCl (pH=6.7) were used. All dialysis were carried out at 4° C., at which leakage of the agent from liposomes is much slower than leakage through the dialysis bag. Under these conditions the level of extraliposomal ammonium sulfate was detected until forming the desired gradients of [(NH₄)₂SO₄]ₗᵢₚ/[(NH₄)₂SO₄]_{med} being in the range of 100-3000.

TMN Loading

After creation of the desired gradient, TiN, BUP or AO were added to all liposomal dispersions at a concentration of 5 mM. Loading was performed at 25° C. (above the Tₘ of the matrix lipid, egg PC) and loading efficiency was determined using the cyclic voltammetry (CV) method, as described hereinafter.

Kinetics of TMN Leakage

Kinetics of agent release from liposomes at 4° C., 25° C., and 37° C. was determined using the CV method, as described hereinafter.

Results

Formation of Aggregates

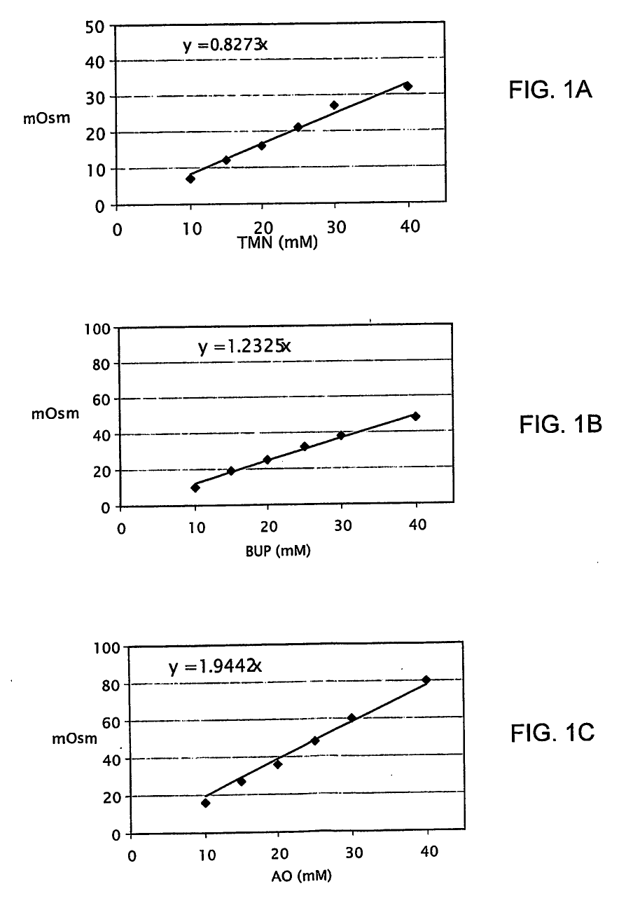

TMN, BUP, and AO osmolality calibration curves in DDW (1040 mM) are presented in FIG. 1. The agents at 25 mM presented the following osmolalities:

TMN—21 mOsm

BUP HCl—32 mOsm

AO—48 mOsm

The effect of salts on the osmotic pressure of the amphipathic base (25 mM) was also examined by comparing the effect of NaCl with that of various ammonium salts. As a control, the osmotic pressure of each salt alone was also measured.

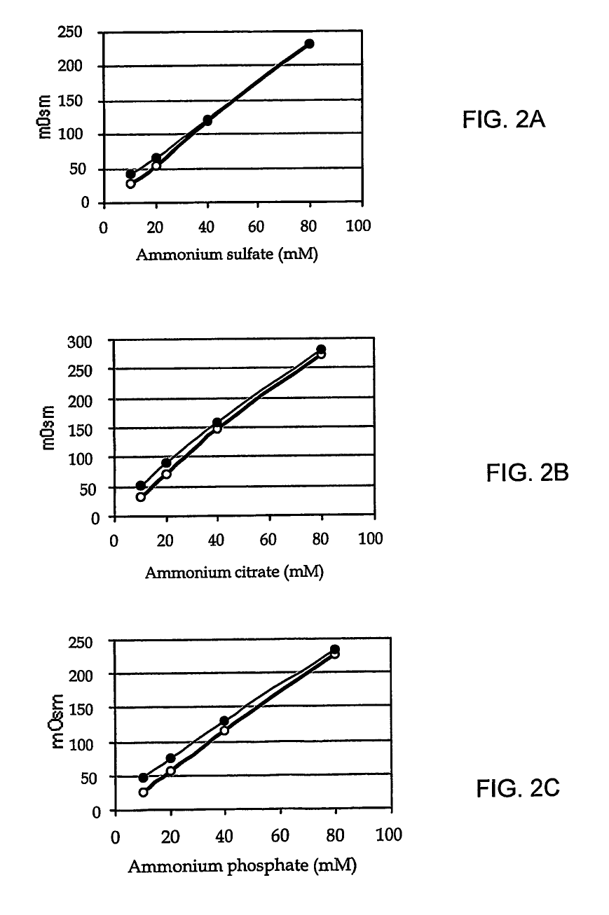

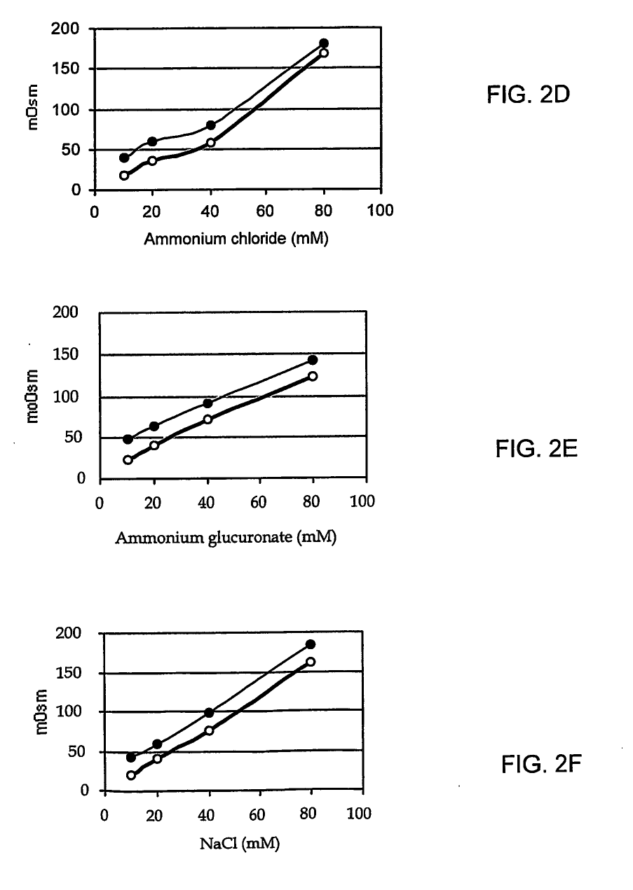

TMN: An osmolility value of 21 mOsm was obtained for TMN in pure water. With different solutions of NaCl an ideal solution and lack of aggregate formation was observed (as the contribution of TMN to the osmolility was identical to its osmolility in pure water). Similar experiments were performed for the various ammonium salts the results of which are shown in FIGS. 2A-2F. As shown, TMN has a different effect on the osmolality of the different salts. The difference in the osmolality was more apparent with higher ammonium salt concentrations, e.g. 80 mM as compared to 40 mM of the ammonium salt, as shown in the following Tables Ia and Ic for the different types of ammonium salts. The contribution of BUP or AO to the osmolality of the salts was also examined and is presented in Tables Ib and Ic.

TABLE Ia
Contribution of 25 mM TMN to osmotic
pressure of ammonium salts

| | Expected | Measured | |
|---|---|---|---|
| | contribution | contribution | |
| | of 25 mM TMN | of 25 mM TMN | |
| Ammonium salt | (calibrated in | (40 mM//80 mM | Level of |
| (40 mM//80 mM) | DDW and NaCl) | ammonium salt) | aggregation |
| Sulfate (SO₄²⁻) | 21 | 4//0 | High//complete |
| Citrate³⁻ | 21 | 13//5 | Medium//high |
| Phosphate | 21 | 15//5 | Medium//high |
| (HPO₄²⁻) | | | |
| Chloride (Cl⁻) | 21 | 21//12 | Low//medium |
| Glucuronate⁻ | 21 | 20//19 | None |

TABLE Ib
Contribution of 25 mM BUP and AO (BUP//AO)
to osmotic pressure of ammonium salts

| | Expected | | |
|---|---|---|---|
| | contribution | | |
| | of 25 mM | Measured | |
| | BUP//AO | contribution | |
| Ammonium salt | (calibrated in | of 25 mM | Level of |
| (80 mM) | DDW and NaCl) | BUP//AO | aggregation |
| Sulfate (SO₄²⁻) | 32//48 | 29//44 | None//Low |
| Citrate³⁻ | 32//48 | 25//43 | None//Low |
| Phosphate(HPO₄²⁻) | 32//48 | 30//41 | None//\|Low |
| Chloride (Cl⁻) | 32//48 | 30//51 | None//None |
| Glucuronate⁻ | 32//48 | 31//50 | None//None |

TABLE Ic
Contribution of amphipathic bases (25 mM) to osmolality
of ammonium salts solutions (40 mM vs. 80 mM)

| Ammonium | TMN (mOsm) | AO (mOsm) | BUP (mOsm) |
|---|---|---|---|
| salt | 40 mM//80 mM | 40 mM//80 mM | 40 mM//80 mM |
| Sulfate | 4//0 (19//0)^{a} | 45//41 (94//85)^{a} | 31//28 (97//88)^{a} |
| (SO₄²⁻) | | | |
| Citrate³⁻ | 13//5 (62//20)^{a} | 46//43 (96//90)^{a} | 32//29 (100//91)^{a} |
| Phosphate | 15//5 (71//20)^{a} | 44//38 (92//79)^{a} | 30//27 (97//84)^{a} |
| (HPO₄²⁻) | | | |
| Chloride (Cl⁻) | 21//12 (100//60)^{a} | 49//48 (100//100)^{a} | 31//32 (97//100)^{a} |
| Glucuronate⁻ | 20//19 (95//90)^{a} | 48//48 (100//100)^{a} | 33//32 (100//100)^{a} |

^{a}% of expected value - percentage from the osmolality of an amphipathic base obtained in pure water or as a contribution to NaCl solutions (these values were found to be identical). Expected osmolaltiy for TMN - 21 mOsm; AO - 48 mOsm, and BUP - 32 mOsm. A decrease in an expected value indicates aggregation.

Based on the results presented in Tables Ia, Ib and Ic, the extent of aggregation of the ammonium salts with TMN was ranked: ammonium sulfate>ammonium citrate=ammonium phosphate>>ammonium chloride>ammonium glucuronate, which shows that ammonium sulfate results in the highest level of aggregation, while ammonium glucuronate did not result in TMN aggregation or precipitation.

Other factors such as conductivity (ionic strength) which may relate to aggregation of amphiphatic weak substances such as TMN with a salt were examined. In addition the extent of dissociation (from the slope of osmolality (mOsm) versus concentration (mM)) of the salts was determined. The results are presented in the following Table II.

TABLE II
Conductivity and slope of osmolality (mOsm) versus
concentration (mM) of different salts (50 mM)

| | Conductivity | mOsm versos mM |
|---|---|---|
| Salts | (μS/cm) | (slope) |
| NaCl | 6.20 | 2.00 |
| (NH₄)₂SO₄ | 9.80 | 2.97 |
| (NH₄)₂HPO₄ | 8.06 | 2.83 |
| (NH₄)₃ citrate | 11.96 | 3.45 |
| NH₄Cl | 5.33 | 1.96 |
| NH₄ glucuronate | 3.88 | 1.62 |

Table II shows that ammonium sulfate, has the highest extent of dissociation among the tested ammonium salts and its conductivity is higher than that of ammonium hydrogen phosphate, the latter having the same number of ions as the ammonium sulfate. Further, the results show that ammonium glucuronate possesses the lowest extent of dissociation and the lowest conductivity. These results conform with the results obtain above, showing that ammonium sulfate possesses the highest ability to aggregate TMN, while ammonium glucuronate does not substantially aggregate TMN.

Another factor that may influence the capability of an ammonium salt to aggregate TMN may be the charge of the anion of the ammonium salt. Glucuronate and chloride, which are monovalent, were found to possess a lower ability to aggregate TMN as compared to the bivalent phosphate and sulfate, or other trivalent citrate. However the bivalent sulfate containing salt was more effective in aggregating TMN than the trivalent citrate.

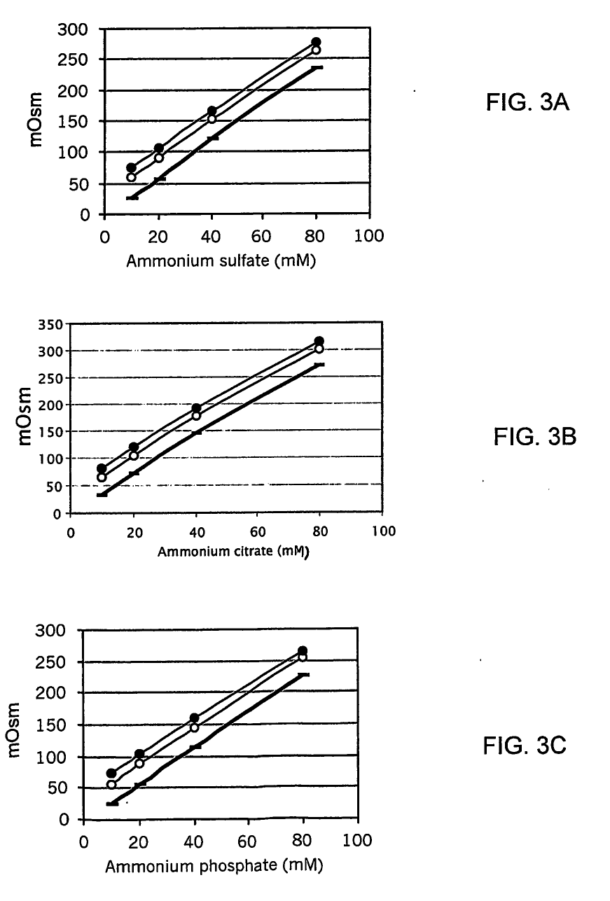

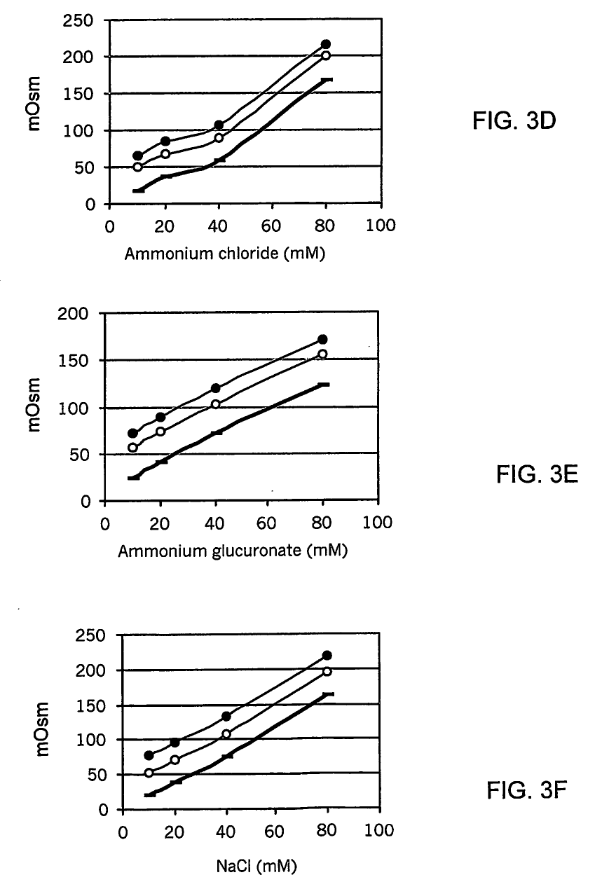

With respect to BUP and AO, the expected contribution to osmolality of 25 mM BUP or AO was 32±5 and 48±4 mOsm, respectively, (calculated from the calibration curve in DDW and in NaCl solutions) was only slightly decreased in ammonium phosphate, ammonium citrate, and ammonium sulfate. Ammonium glucuronate and ammonium chloride did not cause substantial aggregation of AO and BUP, as their contributions to osmolality of these salts was equal to the expected values. These results indicate lack of significant aggregation of BUP and AO, at least up to 25 mMK in the presence of the tested ammonium salts in the range of up to 80 mM. FIG. 3 presents the contribution of 25 mM AO and 25 mM BUP to the osmolality of the different ammonium salts. These results suggest that for aggregation of BUP or AO, other salts should be used.

Notwithstanding the above, BUP and AO aggregation was achieved by adding dextran sulfate (DS) of 8,000 Da (on the average 20 units of glucose-sulfate per molecule 2.3-SO₄ moieties per glucose) or 10,000 Da (on average 25 units of glucose sulfate per one molecule) molecular weight to the medium. Complete aggregation of BUP-DS or AO-DS was obtained starting at a mole ratio of weak base/DS of 1.0 and reached a plateau at a ratio of 50 nmole AO or BUP per 1 mmole DS (10,000 Da).

Dependence of Loading Efficiency and Kinetics of TMN Leakage on Intraliposomal Ammonium Salt Concentration and on Formation of Aggregate.

TMN at a final concentration of 5 mM was added to an MLV (1200±200 nm) suspension composed of egg PC (120 mM phospholipid) after creation of the required ammonium salt gradient. The loading efficiency was dependent on the concentration of the intraliposomal ammonium salt, such that a high level of encapsulation was achieved at higher salt gradients. However, encapsulation efficiency was not dependent on the type of anion derived from the ammonium salt, i.e., it was independent of extent of aggregation (Table III).

TABLE III
Percent encapsulation and TMN to phospholipid mole ratio (TMN/PL)
by gradient induced loading of different ammonium salts

| | Percent encapsulation (TMN/PL) | | |
|---|---|---|---|
| Concentration | Ammonium | Ammonium | Ammonium |
| (mM) | sulfate | glucuronate | chloride |
| 100 | 68% (0.027) | 60% (0.024) | 62% (0.025) |
| 200 | 85% (0.034) | 67% (0.027) | 81% (0.032) |
| 400 | 86% (0.034) | 71% (0.028) | 85% (0.034) |

These results support the statement proposed by Clerc and Barenholz [Clerc and Barenholz (1998); ibid.] that the pH gradient (in this particular case, the ammonium gradient) is a main factor in determining encapsulation efficiency of a weak amphipathic amine.

Kinetics of Leakage

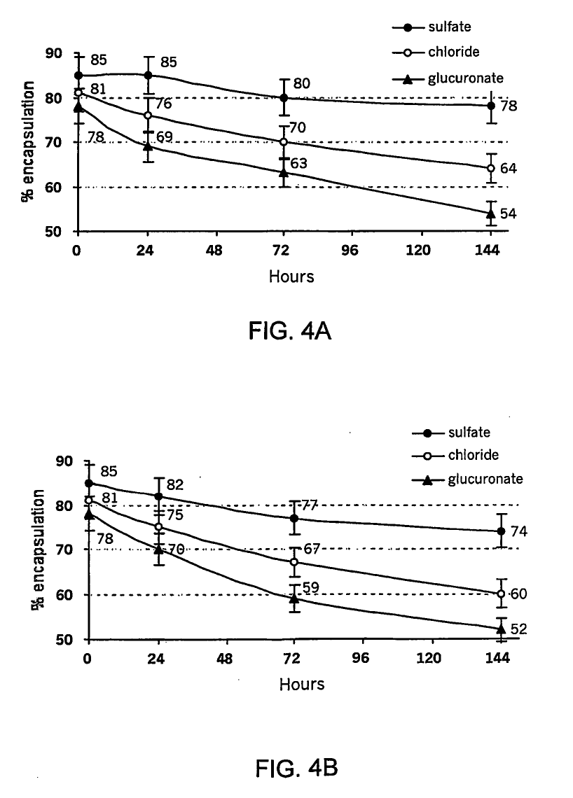

TMN leakage was determined at three temperatures: 4° C., 25° C., and 37° C. Temperature and the type of ammonium salt were found to be important in controlling the stability of the encapsulated TMN. FIG. 4 presents the stability of TMN loaded into egg PC ULV (1200±200 nm) at 25° C. (FIG. 4A) and 37° C. (FIG. 4B). At 4° C., no TV leakage was observed in liposomes loaded via the ammonium sulfate gradient until the last time point at 144 hr (almost 1 week), while in liposomes having ammonium glucuronate and ammonium chloride gradients, leakage at 4° C. did occur, though it was less than 10%. At elevated temperatures (25° C. and 37° C.) the order of encapsulation stability was: (NH₄)₂SO₄>>NH₄Cl>NH₄ glucuronate, strengthening the assumption that extent of aggregation has a strong correlation with the extent of leakage process.

Since no correlation was obtained between the type of ion and anion permeability coefficient (dextran-SO₄²⁻ (10⁻¹¹)<SO₄²⁻ (<10⁻¹² cm/s)≈glucuronate <<Cl⁻ (7.6×10⁻¹¹ cm/s), it may be concluded that permeability of all these anions through the phospholipid membrane is slow enough, thus having substantially no effect on the stability of encapsulated liposome.

The magnitude of the gradient at the tested concentrations (range 100-400 mM), also did not influence the stability of loading, i.e. leakage rate of TMN was similar with each of the four salts at all the gradients (data not shown). This suggests that maximum possible aggregation was achieved already at 100 mM intraliposomal ammonium salt concentration.

Discussion

Many parameters may influence the proton and other ion (i.e., NH₄⁺) gradient driven and loading efficiency of an active agent in liposomes. The present invention discloses a new parameter for controlling the relates rate of an agent fro a liposome and is related to the extent to which the loaded substance aggregates/precipitates in the intratiposomal aqueous phase (the ratio of aggregated/non-aggregated agent). The above, non-limiting examples, show that a release profile of an agent is strongly dependent on the physical state of the molecule inside the liposomes. Therefore the release rate of the molecule from the liposome may be modified by changing its state of aggregation in the intraliposomal aqueous phase.

The ability of the loaded active agent to aggregate inside the liposome may depend on the properties of the active agent, as well as on the composition of the intraliposomal medium. In the above, non-limiting examples, the release profile of three different molecules, TMN, BUP, and AO, was evaluated. As these three compounds are all amphipathic amines their loading was achieved through an ammonium sulfate gradient. In particular, the aggregation of these compounds in solutions of NaCl and in 5 different ammonium salts; in the presence and absence of charged polymers (such as dextran sulfate); their extent of dissociation; their ionic strength, and strength of the acid which contributes the anion were quantified. None of the 3 amphipathic bases significantly aggregated in NaCl. Nevertheless, a correlation between the composition of the salt formed with the amphipathic bases and the ability of the salt formed to aggregate was determined. This correlation show that the interaction between the specific active agent and the counter ion dictates the level of aggregation, and thereby the release profile of the agent from the liposome.

Ammonium sulfate was the most potent in its ability to aggregate TMN, while ammonium phosphate was the best in its ability to aggregate AO and BUP. Extent of ammonium salt dissociation correlated with ability of salt to aggregate TMN. The difference is significant when comparing the salt with the lowest extent of dissociation, ammonium glucuronate, to the salt with the highest extent of dissociation, ammonium sulfate. The former did not cause substantial aggregation of TMN, while the latter gave a very good aggregation and thereby stability (i.e. sustained release).

Anionic charge may also play a role in the aggregation process. Univalent small (nonpolymeric) anions (glucuronate and chloride) were found to posses a lower ability to aggregate TMN than bivalent or trivalent anions, although addition of polymeric counter ion such as dextran sulfate increased the level of aggregation in the case of AO and PUB.

The dependence of the aggregation process on the loaded molecule was shown by the examples of BUP and AO. Neither extent of dissociation of the ammonium salt nor the charge of the anion influenced the aggregation of BUP and AO, suggesting that the chemical properties of the loaded agent also play a role in aggregation. This however can be modified through the use of sodium salt of dextran sulfate as an additional counter ion. Although the charged polymer is highly soluble in aqueous solution when it associates, for example with AO, precipitation of AO-DS started to occur at a ratio of 1 nmole AO/1 nmole DS and reaches a saturation at a ratio of 40 for DS 8,000 Da and 50 for DS 10,000 Da.

The extent of TMN aggregation in the intraliposomal aqueous phase was found to influence its release kinetics. Three ammonium salts were evaluated for their ability to aggregate TMN: ammonium sulfate, ammonium chloride, and ammonium glucuronate, which showed very different aggregation results, high aggregation low aggregation and no aggregation, respectively.

Therefore, it was concluded that aggregation plays an important role in controlling kinetics of agent release from liposomes; a high extent of aggregation provides a relatively slower rate of active agent release. Thus, it is possible, by the selection of a counter ion to design a release profile of an agent according to the needs, the release profile being dependent on the determination of the extent of aggregation of the agent inside the liposome, also expressed as a mole ratio of aggregated/nonaggregated agent.

Other parameters that may influence kinetics of release include the lipophilicity of the loaded agent and the liposome trapped volume. The higher the lipophilicity, the faster will be the release rate of the agent from the liposome. The lipophilicity of a compound may be reflected by its partition coefficient (Kₚ) is between octanol and water. Table IV shows partition coefficients of the 3 tested agents.

TABLE IV
Partition coefficient (Kₚ) octanol/water

| Compound | Kp |
|---|---|
| TMN | 0.04 (at pH 7, 150 mM (NH₄)₂SO₄) |
| BUP | 0.35 (at pH 6, 250 mM (NH₄)₂SO₄) |
| AO | 9.00 (at pH 5.5, 120 mM (NH₄)₂SO₄) |

TMN is the least lipophilic of the three bases, which may be another factor for its low leakage rate, in addition to aggregation.

While passive loading of an agent increases when trapped volume is increased, this is not always the case for active loading by ammonium sulfate. In this case the loading efficiency increases with reduction in liposome size. This is explained by the following: the smaller the liposome intraliposome aqueous phase, the smaller (and therefore easier) is the number of ions that have to move across the membrane in order to form the efficient gradients ammonium sulfate (e.g., for weak bases) or calcium acetate (e.g., for weak acids) and therefore to induce intraliposome loaded drug precipitation which correlate with loading stability and with rate of agent release. Therefore size of liposome may be considered as another mean to modulate their release profile.

The invention will now be defined by the appended claims, the contents of which are to be read as included within the disclosure of the specification.

1. A method for preparing a liposomal formulation for delivery of an active agent to a target, the release of said active agent from the liposome being designed to have a release profile in which the release is sustained for a time period to achieve an optimal effect of the active agent at said target, the method comprising
preparing a liposomal formulation, wherein the liposome is loaded with said active agent, and with a selected counter ion, said ion and said active agent interacting together to aggregate and/or to form a precipitate within the liposome, wherein the counter ion being selected such that the release of the active agent from the liposome has said release profile.

2. The method of claim 1, wherein said release profile is a controlled release profile and is dependent on the extent of interaction between of the active agent and the counter ion.

3. The method of claim 2, wherein said release profile is a slow release profile and said counter ion is selected such that its interaction with said active agent forms within the liposome a substantially water insoluble salt.

4. The method of claim 2, wherein said release profile is affected by the charge of the selected counter ion.

5. The method of claim 1, wherein said active agent is a weak amphipathic base or a weak acid.

6. The method of claim 1, wherein said active agent and said counter ion is encapsulated within the liposome by the formation of a pH or ion gradient, the counter ion being obtained from the ion gradient or by its passive loading into the liposome.

7. The method of claim 6, wherein said active agent is a weak amphipathic base and the pH gradient is formed by the use of an ammonium salt.

8. The method of claim 7, wherein said counter ion is derived from an ammonium salt forming the pH gradient.

9. The method of claim 1, wherein said counter ion is selected from the group consisting of sulfate, borate, phosphate, citrate, chloride, glucuronate, hydroxide, carbonate, bicarbonate, nitrate, cyanate, acetate, benzoate and bromide.

10. The method of claim 1, wherein said active agent is a weak amphipathic acid and said counter ion is selected from the group consisting of calcium, magnesium, sodium, ammonium.

11. The method of claim 1, wherein said active agent is a weak amphipathic base and said counter ion is an anionic polymer.

12. The method of claim 11, wherein said anionic polymer comprises, covalently attached to a polymeric moiety, a counter ion selected from sulfate, phosphate, carbonate, borate citrate, carboxymethyl dextran.

13. The method of claim 1, wherein said active agent is a weak amphipathic acid and said counter ion is a cationic polymer.

14. The method of claim 13, wherein said cationic polymer comprises, covalently attached to a polymeric moiety, a counter ion selected from calcium, magnesium, sodium, ammonium.
